(19) United States
(12) Patent Application Publication
(10) Pub. No.: US 2021/0353806 A1
(43) Pub. Date: Nov. 18, 2021

(54) ELEVATOR STERILIZATION SYSTEM AND ASSOCIATED METHODS

(71) Applicant: Healthe, Inc., Cocoa Beach, FL (US)

(72) Inventor: Fredric Maxik, Cocoa Beach, FL (US)

(21) Appl. No.: 17/320,463

(22) Filed: May 14, 2021

Related U.S. Application Data
(60) Provisional application No. 62/704,523, filed on May 14, 2020.

(51) Int. Cl. A61L 2/24; A61L 2/10

(52) U.S. Cl. CPC A61L 2/24; A61L 2/10; A61L 2202/25; A61L 2202/14; A61L 2202/11

(57) ABSTRACT

A sterilization device for deactivating pathogens within an elevator cabin is disclosed. The sterilization device comprises a housing, one or more ultraviolet (UV) light sources, a sensor, a processor, and a non-transitory, computer readable medium. The housing defines an interior space containing the UV light sources and an aperture allowing the UV light sources to emit UV light to a coverage zone in the elevator cabin. The sensor is configured to detect a living presence within the coverage zone. Based on instructions stored by the computer readable medium, the processor cycles the UV light sources on a first schedule while the sensor indicates that the living presence is in the coverage zone and cycles the UV light sources on a second schedule for a set period of time once the sensor indicates that the living presence is no longer in the coverage zone. Thereafter, the UV light sources are deactivated.

### CROSS-REFERENCE TO RELATED APPLICATIONS

The application claims the benefit of priority to U.S. Provisional Application No. 62/704,523 entitled “Elevator Sterilization System and Associated Methods,” filed May 14, 2020, which is incorporated herein by reference in its entirety.

### TECHNICAL FIELD

The present invention relates generally to devices and systems for sterilization an elevator using ultraviolet (UV) light. More particularly, the device may emit UV light to an elevator cabin to irradiate air and surfaces within the field of the device. The disclosed devices and systems may be applied to sanitize air within an elevator cabin, surfaces of the elevator cabin, and objects and/or clothing within the elevator cabin in a selective manner.

### BACKGROUND

The impact of the spread of viruses has been acutely felt throughout the world in the present time. COVID-19, SARS, and other viruses and microorganisms have had a significant and deadly impact on the way that individuals live their lives. In particular, individuals are less willing and/or able to occupy public spaces, such as malls, restaurants, theaters, public transit states, event and conference spaces, and other crowded locations, for fear of being exposed to and succumbing to a virus.

In order to combat the spread of viruses in public spaces, various precautions have been implemented. Due to the airborne nature of many pathogens including COVID-19, covering one's face with a fabric mask and maintaining physical distance from others is recommended. Proper sanitization of surfaces, especially those that experience frequent human contact, may also be crucial to reduce transmission of pathogens. However, standard cleaning protocols and routines may not efficiently remove pathogens to the degree necessary to significantly impact human-to-human transmission.

More recently, ultraviolet light has been introduced as a means to sanitize surfaces and substances. The type of ultraviolet (UV) light has been classified into at least four bands depending upon the effects upon the skin of humans and other animals. Such bands include UV-A, which is defined as ultraviolet light having a wavelength in a range from 315 nm to 400 nm; UV-B, which is defined as ultraviolet light having a wavelength in a range from 280 nm to 315 nm; UV-C, which is defined as ultraviolet light having a wavelength that is in a range from 235 nm to 280 nm; and Far UV, which is defined as ultraviolet light having a wavelength that is in a range from 185 nm to 235 nm.

Ultraviolet light in the UV-C range has been used for sanitization. For example, UV light emitted at 254 nm and 265 nm has been used to destroy viruses and other microorganisms for a number of years. Far UV light (e.g., 222 nm) has been shown to have some efficacy for this use as well. However, UV light emitted in the UV-C range can have harmful impacts on humans. For example, prolonged direct exposure to UV-C light can result in eye and skin damage, such as acute corneal injury (sometimes referred to as “welder's eye”) and acute erythema. Acute effects from UV-C light include redness, ulceration or burns of the skin. Longer-term effects may include premature aging of the skin and/or skin cancer.

Sanitization of objects and/or clothing may be beneficial upon entry to or egress from private or shared public spaces. Accordingly, a contained entry and/or egress space such an as elevator may benefit from implementation of a sanitization procedure to promote or ensure hygiene within the space and/or sanitization upon egress from any pathogens present within the space. While sanitizing with chemicals, wipes, and other cleanings products may be effective, the frequency of ingress and egress of individuals and objects may render such an approach infeasible. Further, although UV sanitization may be suitable for this purpose, a continuously running UV sanitization system may have restrictively large power requirements and/or may be harmful to humans.

As such, it would be desirable to have a sterilization system that uses UV light to sterilize air, surfaces, and objects within an elevator cabin and may be regulated based on human presence.

### SUMMARY

A sterilization device for deactivating pathogens within an elevator cabin is provided. The sterilization device comprises a housing, one or more ultraviolet (UV) light sources, one or more sensors, a processor, and a non-transitory, computer-readable medium. The housing defines an interior space and an aperture communicating with the interior space. The one or more UV light sources are arranged in the interior space and configured to emit UV light to a coverage zone within the elevator cabin via the aperture to deactivate pathogens in the coverage zone. The one or more sensors are configured to detect a living presence within the coverage zone. The non-transitory, computer-readable medium stores instructions that, when executed, cause the processor to: receive a first detection signal from the one or more sensors indicative of a living presence within the coverage zone, operate the one or more UV light sources to emit UV light to the elevator cabin according to a first schedule in response to the one or more detection signals, receive a second detection signal from the one or more sensors indicative of an absence of the living presence within the coverage zone, operate the one or more UV light sources to emit UV light to the elevator cabin according to a second schedule for a predetermined period of time after a first time delay from receiving the second detection signal, and deactivate the one or more UV light sources after the predetermined period of time.

According to some embodiments, the one or more UV light sources are configured to emit a sanitizing dose of UV light to the coverage zone within the predetermined period of time, wherein the sanitizing dose is configured to deactivate pathogens within the coverage zone.

According to some embodiments, the one or more sensors comprise a passive infrared sensor configured to detect motion of the living presence.

According to some embodiments, the housing further comprises one or more spring-loaded mounting clips configured to affix the housing within a light socket of an elevator cabin.

According to some embodiments, the instructions that cause the processor to operate the one or more UV light sources according to a first schedule comprise instructions that, when executed, cause the processor to cycle the one or more UV light sources between a first period of about 70 seconds of emission and a second period of about 30 seconds of inactivity until the one or more light sources emit UV light for a total emission time. According to additional embodiments, the total emission time is selected from the group consisting of about 12 minutes, about 18 minutes, about 24 minutes, and about 30 minutes. According to additional embodiments, the sterilization device further comprises an input device configured to receive input from a user related to the total emission time. In some embodiments, the instructions, when executed, further cause the processor to: receive the input from the user via the input device, and set the total emission time based on the input.

According to some embodiments, the instructions that cause the processor to operate the one or more UV light sources according to a second schedule comprise instructions that, when executed, cause the processor to cycle the one or more UV light sources between a first period of about 70 seconds of emission and a second period of about 30 seconds of inactivity until the one or more light sources emit UV light for a total of about 60 minutes during the predetermined time period. According to additional embodiments, the predetermined period of time is about 86 minutes.

According to some embodiments, the one or more UV light sources are selected from the group consisting of light-emitting diodes, mercury vapor discharge devices, laser diodes, pulsed xenon lasers, fiber lasers, and excimer lamps.

According to some embodiments, the sterilization device further comprises one or more visible light sources disposed on the housing and configured to emit visible light to the elevator cabin. According to additional embodiments, the sterilization device further comprises a translucent cap coupled to the housing over the one or more visible light sources. In some embodiments, the visible light from the one or more visible light sources is configured to diffuse through the translucent cap and to the elevator cabin. According to additional embodiments, the instructions, when executed, further cause the processor to: activate the one or more visible light sources to emit the visible light in response to the one or more detection signals, and, after a second time delay from receiving the second detection signal, deactivate the one or more visible light sources. According to further embodiments, the first time delay is substantially equal to the second time delay.

An alternate sterilization device for deactivating pathogens within an elevator cabin is also provided. The sterilization device comprises a housing, one or more ultraviolet (UV) light sources, one or more sensors, a processor, and a non-transitory, computer-readable medium. The housing defines an interior space and an aperture communicating with the interior space. The one or more UV light sources are arranged in the interior space and configured to emit UV light to a coverage zone within the elevator cabin via the aperture to deactivate pathogens in the coverage zone. The one or more sensors are configured to detect a living presence within the coverage zone. The non-transitory, computer-readable medium stores instructions that, when executed, cause the processor to: receive a first detection signal from the one or more sensors indicative of a living presence within the coverage zone, maintain the one or more UV light sources in a deactivated state in response to the one or more detection signals, receive a second detection signal from the one or more sensors indicative of an absence of the living presence within the coverage zone, activate the one or more UV light sources to emit UV light to the elevator cabin according to a schedule for a predetermined period of time after a first time delay from receiving the second detection signal, and deactivate the one or more UV light sources after the predetermined period of time.

According to some embodiments, the sterilization device further comprises one or more visible light sources disposed on the housing and configured to emit visible light to the elevator cabin, and a translucent cap coupled to the housing over the one or more visible light sources. In some embodiments, the visible light from the one or more visible light sources is configured to diffuse through the translucent cap and to the elevator cabin. According to additional embodiments, the instructions, when executed, further cause the processor to: activate the one or more visible light sources to emit the visible light in response to the one or more detection signals, and, after a second time delay from receiving the second detection signal, deactivate the one or more visible light sources.

According to some embodiments, the first time delay is substantially equal to the second time delay.

According to some embodiments, the instructions that cause the processor to operate the one or more UV light sources according to a schedule comprise instructions that, when executed, cause the processor to cycle the one or more UV light sources between a first period of about 70 seconds of emission and a second period of about 30 seconds of inactivity until the one or more light sources emit UV light for a total of about 60 minutes during the predetermined time period. According to additional embodiments, the predetermined period of time is about 86 minutes.

An elevator sterilization system for deactivating pathogens is also provided. The elevator sterilization system comprises an elevator cabin, one or more sterilization devices, one or more sensors, a processor, and a non-transitory, computer readable medium. The elevator cabin includes one or more doors. The one or more sterilization devices each comprise a housing and one or more ultraviolet (UV) light sources. The housing defines an interior space and an aperture communicating with the interior space. The one or more ultraviolet (UV) light sources are arranged in the interior space and configured to emit UV light to a coverage zone within the elevator cabin via the aperture to deactivate pathogens in the coverage zone. The one or more sensors are configured to detect a living presence proximate the elevator cabin. The non-transitory, computer readable medium stores instructions that, when executed, cause the processor to: receive one or more first detection signals from the one or more sensors indicative of a living presence within the coverage zone of a first set of the one or more sterilization devices, operate the one or more UV light sources of the first set to emit UV light to the elevator cabin according to a first schedule in response to the one or more first detection signals, receive one or more second detection signals from the one or more sensors indicative of an absence of the living presence within the coverage zone of the first set, operate the one or more UV light sources of the first set to emit UV light to the elevator cabin according to a second schedule for a predetermined period of time after a first time delay from receiving the second detection signal, and deactivate the one or more UV light sources after the predetermined period of time.

According to some embodiments, each sensor is associated with a sterilization device of the one or more sterilization devices. In some embodiments, the sensor is configured to detect the living presence within the coverage zone.

According to some embodiments, the instructions that cause the processor to operate the one or more UV light sources of the first set according to a first schedule comprise instructions that, when executed, cause the processor to cycle the one or more UV light sources of the first set between a first period of about 70 seconds of emission and a second period of about 30 seconds of inactivity until the one or more light sources emit UV light for a total emission time. In some embodiments, the total emission time is selected from the group consisting of about 12 minutes, about 18 minutes, about 24 minutes, and about 30 minutes.

According to some embodiments, the system further comprises a frame comprising a plurality of recesses. In some embodiments, each recess is configured to receive a sterilization device of the one or more sterilization devices to affix the sterilization device to the frame.

According to some embodiments, at least one of the one or more sensors comprise one or more light sensors configured to detect one or more changes in ambient light in the elevator cabin associated with opening of the one or more doors. In some embodiments, the one or more first detection signals and the one or more second detection signals are associated with the one or more changes in ambient light.

According to some embodiments, each sterilization device further comprises one or more visible light sources disposed on the housing and configured to emit visible light to the elevator cabin; and a translucent cap coupled to the housing over the one or more visible light sources. In some embodiments, the visible light from the one or more visible light sources is configured to diffuse through the translucent cap and to the elevator cabin. According to additional embodiments, the elevator cabin further comprises a titanium oxide coating on one or more surfaces of the elevator cabin. According to further embodiments, the one or more visible light sources of each sterilization device comprise a blue light pump having a peak wavelength of about 435 nm. In some embodiments, the titanium oxide coating reacts with the visible light from the one or more visible light sources to induce photocatalytic effects on the one or more surfaces.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts a perspective view of an elevator sterilization device for deactivation of pathogens by ultraviolet (UV) radiation in accordance with an embodiment.

FIG. 2 depicts a bottom up view of the elevator sterilization device of FIG. 1 in accordance with an embodiment.

FIG. 3 depicts an elevator sterilization system disposed within an elevator cabin in accordance with an embodiment.

### DETAILED DESCRIPTION

This disclosure is not limited to the particular systems, devices and methods described, as these may vary. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is also to be understood that the terminology used herein is for the purpose of describing particular embodiments only and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the devices, systems, and methods can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

The use of the terms first, second, etc. do not denote any order or importance, but rather the terms first, second, etc. are used to distinguish one element from another.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

Unless defined otherwise, all technical and scientific terms used herein have the same meanings as commonly understood by one of ordinary skill in the art, including scientists, engineers, researchers, industrial designers, manufacturers, technicians, and users of the systems and methods for their designed purposes.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure. Nothing in this disclosure is to be construed as an admission that the embodiments described in this disclosure are not entitled to antedate such disclosure by virtue of prior invention.

### Elevator Sterilization Device

As discussed herein, it would be advantageous to have a sterilization device and/or sterilization system for sterilizing an elevator cabin. As generally described herein, the devices and systems utilize UV light to sterilize air within the elevator cabin and surfaces of the elevator cabin. Further, the devices and systems may sterilize objects within the elevator cabin and/or clothing or other items worn by an individual within the elevator cabin. In some cases, the sanitization system may be regulated based on the detection of human presence.

As shown and described by the various figures and accompanying text, embodiments of an elevator sterilization device that utilize UV radiation to deactivate pathogens within the radiation field of the elevator sterilization device are provided. The elevator sterilization device 100 may be any device operable to emit UV radiation into an elevator environment. For example, the radiation field may include at least a portion of the elevator cabin, i.e., the enclosed volume of an elevator within which passengers and/or cargo may be stored and elevated.

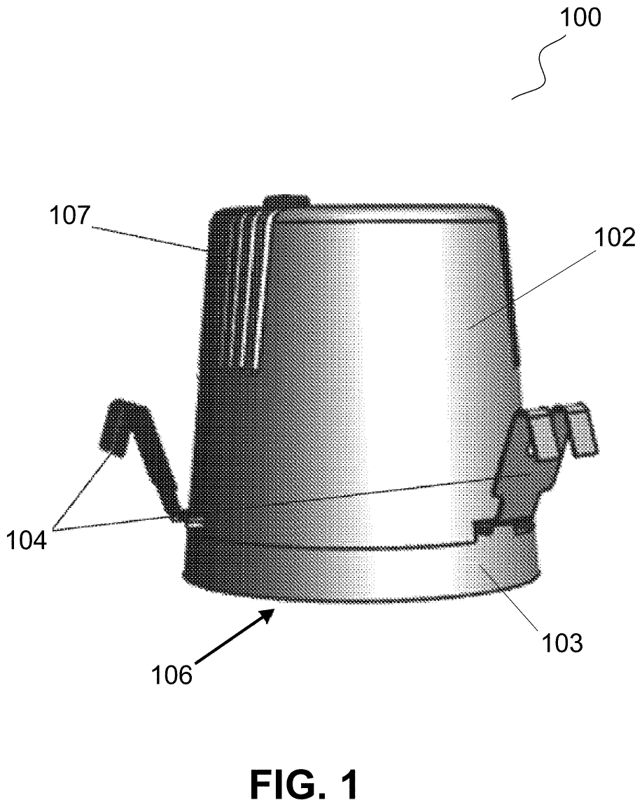

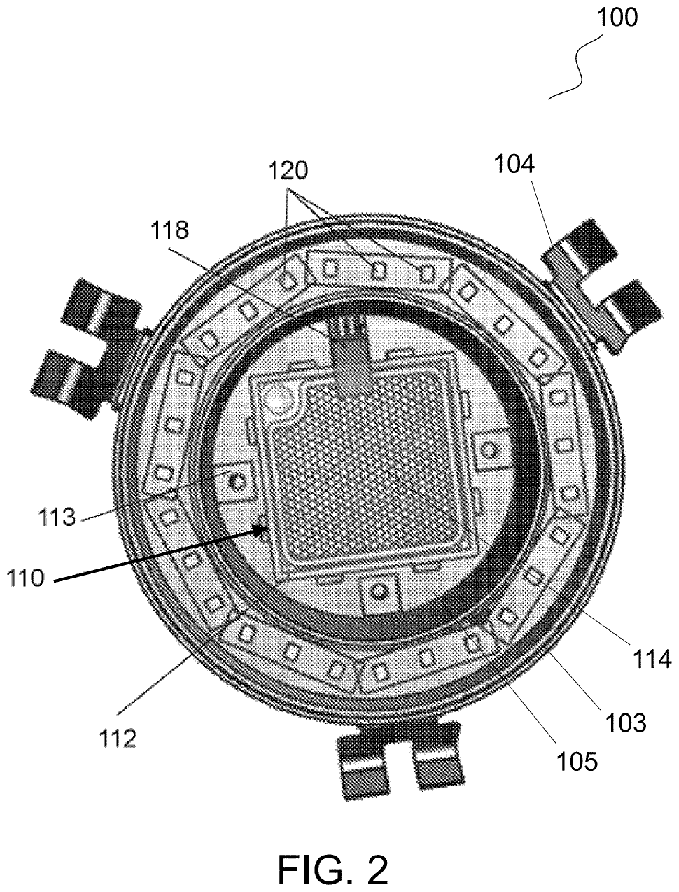

FIG. 1 depicts a perspective view of the elevator sterilization device 100 in accordance with an embodiment. FIG. 2 depicts a bottom up view of elevator sterilization device 100 in accordance with an embodiment. Similar features within FIGS. 1-2 are identified with common reference numbers. The elevator sterilization device 100 may comprise a housing 102, a transparent or translucent cap 103 coupled to an end of the housing 102, spring-loaded mounting clips 104 coupled to at least one of the housing 102 and the transparent cap 103, and one or more radiation assemblies 110 disposed within the housing 102.

In some embodiments, the housing 102 comprises a cylinder or a canister. The housing 102 may comprise an interior space 105 for housing components of the elevator sterilization device 100. Furthermore, the housing 102 and the transparent cap 103 may together define an aperture 106 extending therethrough to provide open access to the interior space 105. In some embodiments, a vent 107 is provided on a portion of the housing 102, e.g., on a side portion as shown in FIG. 1. However, it should be understood that the vent 107 may be disposed at various locations of the elevator sterilization device 100 as would be known to a person having an ordinary level of skill in the art.

In some embodiments, the transparent or translucent cap 103 is coupled to and carried by the housing 102. In some embodiments, the transparent or translucent cap 103 may comprise an opening therein forming at least part of the aperture 106 in order to allow UV light to be emitted directly therethrough. For example, as shown in FIG. 2, the transparent or translucent cap 103 may be ring-shaped.

As shown in FIG. 2, the radiation assembly 110 may be disposed within the interior space 105 of the body and exposed by the aperture 106. For example, the radiation assembly 110 may be positioned in the interior space 105 and oriented such that radiation emitted thereby may pass through the aperture 106 and into a coverage zone of the elevator sterilization device 100, i.e., within an elevator cabin associated with the elevator sterilization device 100. The coverage zone (also referred to as a “field of view” or a “field of coverage”) may be defined as a volume of space over which the radiation assembly 110 delivers a sanitizing dose of radiation as further described herein. Radiation emitted by each radiation assembly 110 may propagate into the elevator cabin to irradiate the air and surfaces within the coverage zone. For example, the radiation may irradiate doors, walls, handrails, floors, control panels, buttons, and the like. Furthermore, the radiation may irradiate surfaces of objects placed within the coverage zone. For example, the radiation may irradiate parcels, luggage, clothing and/or wearable items worn by individuals within the coverage zone, and any additional items placed within the field of coverage. In some embodiments, each elevator sterilization device 100 may be positioned within the elevator such that the radiation assembly 110 is oriented to irradiate a specific areas and/or surfaces. For example, the elevator sterilization device 100 may be arranged to irradiate a particular region of the elevator cabin including walls or regions of walls, regions of the floor, regions of the ceiling, and the like. In some embodiments, the elevator sterilization device 100 comprises a single radiation assembly 110. In some embodiments, the elevator sterilization device 100 comprises a plurality of radiation assemblies 110.

Each radiation assembly 110 may comprise a radiation assembly housing 112 configured to be attached to and carried by the housing 102 by one or more brackets 113, a radiation-emitting device 114 configured to emit radiation through one or more openings in the radiation assembly housing 112, and control circuitry (not shown) in electrical communication with the radiation-emitting device 114 and configured to provide power to and control the operation of the radiation-emitting device 114.

In some embodiments, a radiation-emitting device 114 of the radiation assembly 110 is configured to emit radiation through openings in the radiation assembly housing 112 and out of the aperture 106 of the elevator sterilization device 100. The radiation-emitting device 114 may comprise any device operable to emit radiation within the electromagnetic radiation ranges described herein, including, but not limited to, light-emitting diodes (LEDs), mercury vapor discharge devices, laser diodes (LDs), pulsed xenon lasers, fiber lasers, additional types of lasers, and/or excimer lamps. Additional and/or alternate types of radiation-emitting devices 114 may also be used as will be apparent to a person having an ordinary level of skill in the art based on the teachings of this disclosure.

As described and shown in FIG. 2, the transparent cap 103 has an opening therein forming at least part of the aperture 106 such that the transparent cap 103 does not cover the one or more radiation assemblies 110. For example, as shown in FIG. 2, the transparent cap 103 may be ring-shaped and may extend around the radiation assembly 110. Accordingly, the transparent cap 103 may permit radiation emitted by the radiation-emitting device 114 to pass through the opening in the transparent cap 103 and into the coverage zone within the elevator cabin without diffusing through the transparent cap 103.

As described herein, the radiation-emitting device 114 may be configured to emit radiation to deactivate pathogens within the coverage zone in the elevator. Such radiation may be within specific wavelength ranges and have a specific wavelength with a maximum intensity of radiation emitted by the radiation-emitting device. In some embodiments, the radiation-emitting device 114 may be configured to emit electromagnetic radiation having a peak intensity within the Far UV range, i.e., within a range from 185 nm to 235 nm. In some embodiments, the radiation-emitting device 114 may be configured to emit electromagnetic radiation having a peak intensity within a wavelength range from 217 nm to 227 nm. In some embodiments, the radiation-emitting device 114 may be configured to emit electromagnetic radiation having a peak intensity of 222 nm, which is substantially safe for human and/or animal exposure.

It should be understood that the radiation-emitting devices 114 may provide a fluence (e.g., a combined fluence) that effectively sanitizes surfaces of objects within the coverage zone of viruses, bacteria, and/or other pathogens. The total dose of UV radiation to which objects are exposed may be based on the number of radiation-emitting devices 114, the fluence of the radiation-emitting devices 114 upon the objects, the position and orientation of the radiation-emitting devices 114 with respect to the objects, and the total exposure time during which UV light is emitted. A predetermined dose (i.e., a sanitizing dose) is delivered to the surface of each object in order to effectively sanitize the object. In some embodiments, the elevator sanitization device 100 may be configured to sanitize objects within a specified amount of time. In some embodiments, the elevator sanitization device 100 may be configured to sanitize objects with 15 seconds, 10 seconds, 5 seconds, 4 seconds, 3 seconds, 2 seconds, 1 second, less than 1 second, or individual values or ranges therebetween. Accordingly, the number of radiation-emitting devices 114, the position and orientation of the radiation-emitting devices 114, and the fluence of the radiation-emitting devices 114 may be selected to provide a sanitizing dose within a selected timeframe.

In some embodiments, the control circuitry of the radiation assembly 110 comprises at least one processor (e.g., a microprocessor) and any number of additional electrical components to monitor and control the function of the elevator sterilization device 100. In some embodiments, the control circuitry further comprises a non-transitory, computer readable medium (e.g., a memory component) in communication with the processor and operable to store software and other data therein. The computer readable medium may be both readable and writable by the processor. In some embodiments, the control circuitry comprises an integrated circuit (IC), one or more field programmable gate assemblies (FPGA), and the like. In some embodiments, the control circuitry further comprises a communication device operable to communicate with a remote computerized device either directly or across a network, including personal area networks (PANs), local area networks (LANs), wide area networks (WANs), and any other network as would be known to a person having an ordinary level of skill in the art. The communication device may comprise a wired communication device that transmits and receives information via a universal serial bus (USB), Ethernet, and/or the like, and/or a wireless communication device operable to communicate using any wireless communication standard, including all IEEE 802 standards, including Wi-Fi, Bluetooth, Bluetooth LE, and/or the like.

In some embodiments, each radiation assembly 110 may further comprise one or more sensors 118 positioned in communication with the control circuitry and operable to detect radiation either emitted by or reflected from an object within a field of view of the sensor 118 (e.g., within the elevator cabin or a portion thereof). In some embodiments, a sensor 118 may be configured to detect infrared (IR) radiation resulting from, for example, the body heat of an individual occupying space within the elevator cabin. In some embodiments, one or more of a sensor 118 and the radiation-emitting device 114 may be operable to emit radiation that can reflect off an object occupying the field of view and be detected by the sensor 118 to indicate the object's presence within the field of view. In some embodiments, the field of view of the sensor 118 may substantially overlap with the coverage zone of the radiation assemblies 110 and/or may be congruent with the coverage zone of the radiation assemblies 110. In some embodiments, the field of view of the sensor 118 may be greater than the coverage zone of the radiation assemblies 110 in order to detect objects imminently entering the coverage zone.

In some embodiments, the control circuitry may be configured to receive signals from the one or more sensors 118 and determine the presence or absence of objects and/or living specimens within and/or near the coverage zone. In some embodiments, the control circuitry may be configured to determine whether an object within or near the coverage zone is living or non-living.

For example, the control circuitry may interpret control signals from an infrared sensor 118 to determine whether the object is emitting heat in a manner consistent with the body of a living specimen. In some embodiments, the sensor 118 is a passive infrared (PIR) sensor. However, any method and/or device for detecting objects and/or living specimens are contemplated and intended to be included within the scope of the disclosure. In some embodiments, the sensors 118 may additionally or alternatively comprise a variety of types of sensors from which the presence of living specimens (e.g., humans and/or animals) and/or objects may be determined or inferred. For example, the sensors 118 may additionally or alternatively comprise proximity sensors (e.g., ultrasonic proximity sensors, capacitive proximity sensors, infrared proximity sensors, and/or time-of-flight sensors), motion sensors (e.g., cameras or infrared motion sensors), acoustic sensors, and ambient light sensors. In some embodiments, a combination of types of presence sensors as described may be employed in order to more accurately detect the presence of a human, animal, or object. It should be understood that any sensor capable of detecting an object and/or living specimen as would be apparent to a person having an ordinary level of skill in the art is contemplated and included within the scope of the invention.

As described herein, the radiation-emitting device 114 may be configured to emit electromagnetic radiation having a peak intensity within the Far UV range. In embodiments where the control circuitry is configured to differentiate between living and non-living specimens, the radiation-emitting device 114 may further be configured to emit a second electromagnetic radiation within the UV-C range, e.g., having a peak intensity within a range from 249 nm to 259 nm, and in further embodiments having a peak intensity of 254 nm. The radiation-emitting device 114 may additionally be configured to emit a third electromagnetic radiation having a peak intensity within a range from 260 nm to 270 nm, and in further embodiments to emit electromagnetic having a peak intensity of 265 nm. Accordingly, the control circuitry may be configured to emit the first electromagnetic radiation having a peak intensity at 222 nm when the specimen is determined to be a living organism, and to emit the first, second, and/or third electromagnetic radiations having peak intensities at 222 nm, 254 nm, and/or 265 nm, respectively, upon determining the specimen is not a living organism.

It should be understood that radiation may be emitted beyond the coverage zone as described herein. For example, while the coverage zone defines a region receiving a sanitizing dose of radiation, some radiation may reach beyond the coverage zone at a lower dose. Accordingly, in embodiments utilizing second and third electromagnetic radiations as described, the sensors 118 may also be configured to detect living specimens beyond the coverage zone of the elevator sterilization device 100, i.e., within a predetermined distance of the elevator sterilization device 100 and/or the coverage zone of the elevator sterilization device 100 such as 1 foot, 2 feet, 3 feet, greater than 3 feet, or individual values or ranges therebetween. In some embodiments, the sensors 118 may detect presence only within the coverage zone and additional sensors may be included for the purpose of detecting the presence of a living specimen within the predetermined distance. The predetermined distance may comprise a safe distance, i.e., a distance beyond which the UV light emitted by the radiation-emitting device 114 is substantially non-harmful to humans and/or living specimens. The sensors (e.g., proximity sensors) may be tailored to detect a presence of a living specimen within a danger zone and eliminate or ignore a presence of a living specimen at a greater distance to prevent false detections. For example, a human standing or walking at a sufficient distance from the elevator sterilization device 100 may not be harmed by the second electromagnetic radiation and/or the third electromagnetic radiation. While specific types of sensors may be particularly advantageous for this purpose, any of the sensors described herein and/or additional types of sensors as would be apparent to a person having an ordinary level of skill in the art may be employed to detect presence beyond the coverage zone. Accordingly, the control circuitry may be configured to emit the first electromagnetic radiation having a peak intensity at 222 nm when a living specimen is present within the predetermined distance of the elevator sterilization device 100 and to emit the first, second, and/or third electromagnetic radiations having peak intensities at 222 nm, 254 nm, and/or 265 nm, respectively, when no living specimens are present within the predetermined distance of the elevator sterilization device 100.

In some embodiments, the elevator sterilization device 100 may additionally or alternatively be manually operated. For example, the elevator sterilization device 100 may further comprise an input device, e.g., a button, other tactile user input, and/or a linked wireless input device (e.g., a tablet, a mobile phone, or another computing device) in communication with the control circuitry via the communication device. The control circuitry may be configured to receive a signal from a user via the input device to activate the radiation-emitting device 114. In some embodiments, the input device may be located with the elevator cabin such that it may be operated by passengers. In some embodiments, the input device may be located and operated remotely, e.g., by staff of a building in which the elevator is located. In some embodiments, the input device is an infrared remote controller.

In some embodiments, the control circuitry of the elevator sterilization device 100 may operate to activate and deactivate the radiation-emitting device 114 selectively according to a schedule. In some embodiments, the radiation-emitting device 114 may be activated and deactivated at specific times. In some embodiments, the radiation-emitting device 114 may be activated and deactivated to achieve a threshold amount of irradiation time over a given time period. For example, the radiation-emitting device 114 may be activated for about 15 minutes per hour, about 30 minutes per hour, and the like. The threshold amount of irradiation time may also be calculated per day, per week, and the like. In some embodiments, irradiation may be performed according to the schedule regardless of the presence of living organisms. In some embodiments, irradiation may be interrupted by the presence of living organisms and continued at a later time. Accordingly, the control circuitry may track the total irradiation time for a given time period and selectively activate and deactivate the radiation-emitting device 114.

In a particular embodiment, the elevator sterilization device 100 may operate in a first sterilization mode whereby exposure of a living organism to UV radiation is limited. In some embodiments, the control circuitry may operate the radiation-emitting device 114 according to a first schedule when a living organism is detected. For example, in response to a signal from the sensors 118 indicating a living organism within the coverage zone, the control circuitry may cycle the radiation-emitting device 114 or the UV light sources thereof such that the radiation-emitting device 114 is activated for about 70 seconds during which the radiation-emitting device 114 emits UV light, and subsequently deactivated for about 30 seconds such that the radiation-emitting device 114 is idle or inactive. In some embodiments, the radiation-emitting device 114 may be cycled through these stages until a threshold amount of emission time is reached. The threshold emission time, i.e., the total time that the radiation-emitting device 114 is activated during the first schedule, may be representative of a set or predetermined maximum time for the living organism to be exposed to UV radiation. In some embodiments, the threshold emission time may be set as a rate, e.g., a threshold emission time per hour, such that the threshold resets every hour. For example, the threshold emission time may be about 12 minutes per hour, about 18 minutes per hour, about 24 minutes per hour, about 30 minutes per hour, greater than about 30 minutes per hour, or individual values or ranges therebetween. After the threshold emission time is reached, the radiation-emitting device 114 may be deactivated until the first schedule is complete and/or until 60 minutes of the first schedule have elapsed. In some embodiments, a user may set the threshold emission time using the input device. Accordingly, exposure of the living organism to UV radiation may be limited based on the preferences and requirements of a particular setting.

Furthermore, the control circuitry may operate the radiation-emitting device 114 according to a second schedule when the living organism is no longer detected. For example, in response to a signal from the sensors 118 indicating the living organism is no longer within the coverage zone, the control circuitry may cycle the radiation-emitting device 114 or the UV light sources thereof such that the radiation-emitting device 114 is activated for about 70 seconds during which the radiation-emitting device 114 emits UV light, and subsequently deactivated for about 30 seconds such that the radiation-emitting device 114 is idle or inactive. In some embodiments, the radiation-emitting device 114 may be cycled through these stages until a threshold amount of emission time is reached. For example, the threshold emission time, i.e., the total time that the radiation-emitting device 114 is activated during the second schedule, may be about 60 minutes. Accordingly, the second schedule may require about 86 minutes to complete. The threshold emission time may be representative of a set or predetermined maximum time for sanitizing the elevator cabin thoroughly after the living organism is no longer present.

In another particular embodiment, the elevator sterilization device 100 may operate in a second sterilization mode whereby exposure of a living organism to UV radiation is eliminated. In some embodiments, the control circuitry may operate the radiation-emitting device 114 to maintain the radiation-emitting device in a deactivated state when a living organism is detected. For example, in response to a signal from the sensors 118 indicating a living organism within the coverage zone, the control circuitry may immediately deactivate the radiation-emitting device 114 and/or maintain the radiation-emitting device 114 in a deactivated state. Accordingly, exposure of the living organism to UV radiation may be eliminated. Thereafter, the control circuitry may operate the radiation-emitting device 114 according to the second schedule as described above when the living organism is no longer detected to sanitize the elevator cabin.

In another particular embodiment, the elevator sterilization device 100 may operate in a third sterilization mode wherein a set schedule is maintained regardless of the presence of living organisms. For example, in the third mode, the control circuitry may cycle the radiation-emitting device 114 or the UV light sources thereof such that the radiation-emitting device 114 is activated for about 70 seconds during which the radiation-emitting device 114 emits UV light, and subsequently deactivated for about 30 seconds such that the radiation-emitting device 114 is idle or inactive. In some embodiments, the radiation-emitting device 114 may maintain this rate of emission constantly, thereby resulting in a total emission time of about 42 minutes per hour.

In some embodiments, the user may select and set the sterilization mode of the elevator sterilization device 100 in one of the first sterilization mode, the second sterilization mode, and the third sterilization mode using the input device as described herein.

In some embodiments, the elevator sterilization device 100 further comprises one or more visible light assemblies 120. The visible light assemblies 120 may comprise one or more visible light sources operably connected to the control circuitry. In some embodiments, the one or more visible light sources comprise LEDs. However, the visible light assemblies 120 may include any visible light sources as would be known to a person having an ordinary level of skill in the art. As shown in FIG. 2, the visible light assemblies 120 may be covered by the transparent cap 103. Accordingly, light emitted by the visible light assemblies 120 may pass through the transparent cap 103 and be diffused therethrough. Accordingly, the diffused light may be emitted to the elevator cabin and the surrounding areas to provide illumination to the elevator cabin, e.g., as white light.

In some embodiments, the visible light assemblies 120 may comprise specific shifted wavelengths. For example, a peak wavelength of a blue LED pump used to create white light emitted by the visible light assembly 120 may be shifted to a wavelength of 435 nm. In some embodiments, one or more surfaces within the elevator cabin may be lined or coated with a reactive component, such as TiO₂, to induce photocatalytic effects when the light from the visible light assembly 120 is emitted thereto. Accordingly, the lined or coated surfaces may undergo additional sanitization by the light of the visible light assembly 120 even when the radiation-emitting device 114 is not activated. This may be particularly advantageous for high contact surfaces, e.g., buttons and/or control panels of the elevator. The TiO₂applied to the exterior surfaces may be sourced from or provided in the form of anatase, ilmenite, rutile, and/or other forms and additional or alternative reactive components may be utilized.

In some embodiments the control circuitry may be further configured to control the visible light assemblies 120 based on detecting a living specimen within the coverage zone by the one or more sensors 118 as described. In some embodiments, the control of the radiation-emitting device 114 and/or the visible light assemblies 120 may be performed on a time delay. For example, when the control circuitry does not detect a living specimen for a predetermined period of time, the visible light assemblies 120 may be turned off and then, after a second period of time, the radiation-emitting device 114 may be activated to irradiate the coverage zone. After a third period of time, e.g., corresponding to a sanitizing dose of radiation, the radiation-emitting device 114 may be deactivated. However, the visible light assemblies 120 and the radiation-emitting device 114 may be controlled in additional combinations. In additional embodiments, the visible light assemblies 120 may be placed in a permanent activated state and/or a permanent deactivated state.

In some embodiments, the user may select and set the illumination mode of the elevator sterilization device 100 as permanently activated mode, permanently deactivated mode, and/or detection-controlled mode using the input device as described herein. The user may switch between the modes using the input device based on the preferences and requirements of a particular setting. In some embodiments, the input device allows for all permutations of sterilization modes and illumination modes described herein.

In some embodiments, the one or more visible light assemblies 120 may be operated manually by a separate input device, e.g., a button, a light switch, a wall switch, a dimmer, a slider, or any other type of input device as would be apparent to a person having an ordinary level of skill in the art.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

In some embodiments, the sensors 118 may additionally or alternatively comprise one or more motion sensors to detect the presence and location of an individual within the elevator cabin. In the case of the sterilization device 100 being installed in a structure, e.g., an elevator cabin, the motion sensor 118 may be used to detect the presence of an individual within the elevator and even further to detect the location of individuals within the elevator. Accordingly, the elevator sterilization device 100 may be configured to emit radiation based on a signal received from the motion sensor 118. Further, the elevator sterilization device 100 may also be configured to emit radiation in a specific direction within the elevator. For example, if the motion sensor 118 detects a new presence within the elevator cabin, e.g., a new guest or person has entered the elevator, then the motion sensor 118 may send a signal to the elevator sterilization device 100 to cause radiation to be emitted in only the direction of the newly detected presence within the elevator, e.g., by selectively activating one or more radiation assemblies 110 of a plurality of radiation assemblies of the elevator sterilization device 100. In another example, if the motion sensor 118 detects a new presence within the elevator in addition to an existing presence (i.e., a first person has been in the elevator and a second person now enters the elevator), the motion sensor 118 may send a signal to the control circuitry to cause the elevator sterilization device 100 to emit radiation in the direction of only the newly sensed presence within the elevator, e.g., by selectively activating one or more radiation assemblies 110 of a plurality of radiation assemblies of the elevator sterilization device 100. More information about such motion sensing can be found in U.S. Pat. No. 9,681,108 entitled “Occupancy Sensor and Associated Methods,” issued on Jun. 13, 2017, and U.S. Pat. No. 9,648,284 entitled “Occupancy Sensor and Associated Methods,” issued on May 9, 2017, which are incorporated by reference herein in their entireties.

### Elevator Sterilization System

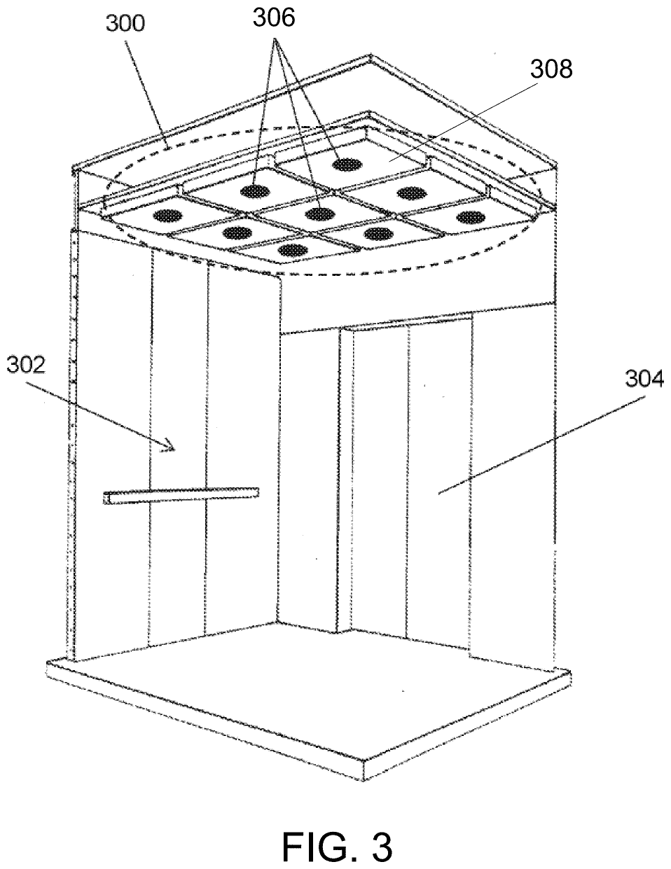

Referring now to FIG. 3, an elevator sterilization system 300 is depicted in accordance with an embodiment. The elevator sterilization system comprises an elevator cabin 302, one or more doors 304, and a plurality of sterilization devices 306 installed within the elevator cabin 302. The sterilization devices 306 may each comprise the elevator sterilization device 100 of FIG. 1 as described herein and may include any of the components, features, and/or characteristics described with respect to the elevator sterilization device 100.

The sterilization devices 306 may be configured to detect a living specimen within or near the coverage zone and to irradiate the coverage zone based on the detection. For example, the sterilization device 306 may comprise sensors (e.g., sensors 118) configured to detect a living specimen and/or may be in electrical communication with a control system of the elevator to receive a signal therefrom indicating presence of a living specimen, e.g., occupancy sensors or weight sensors. In some embodiments, the sterilization devices 306 may operate to emit radiation having a peak wavelength of 222 nm upon detection of a living specimen within the field of view. For example, the sterilization device 306 may activate irradiation with Far UV radiation having a peak wavelength of 222 nm upon entry of an individual into the elevator cabin to irradiate the clothing and or additional objects that have entered the coverage zone. In some embodiments, the sterilization devices 306 may operate to emit radiation after an individual has left the coverage zone. In such embodiments, the sterilization device 306 may emit Far UV and/or UV-C radiation (e.g., 222 nm, 254 nm, and/or 265 nm). In some embodiments, the sterilization devices 306 may emit Far UV radiation upon detection of a living specimen and emit Far UV and/or UV-C radiation when the living specimen is no longer detected.

The sterilization devices 306 and the control circuitry thereof may be configured to emit radiation for a length of time necessary to sterilize the coverage zone, i.e., to provide a sanitizing dose.

In some embodiments, the sterilization devices 306 may receive a signal indicating opening of the doors 304 and/or a signal from a control system of the elevator indicating that the doors 304 are about to be opened. Accordingly, the sterilization devices 306 and the control circuitry thereof may be configured to cease emission of Far UV and/or UV-C radiation upon receiving such a signal. In some embodiments, the sterilization devices 306 may be in electrical communication with the doors 304, a sensor associated with the doors 304, a control system associated with the doors 304 (e.g., a control system of the elevator), and/or the like in order to receive the signal. In some embodiments, the sterilization devices may infer that the doors 304 have opened based on a detected change in ambient light within the elevator 302 by the sensors of the sterilization device 306 (e.g., sensors 118 and/or additional light sensors). Accordingly, the sterilization devices 306 may be configured to operate in different modes based on the indication of the doors 304 and/or the detection of a living specimen.

For example, the sterilization devices 306 may emit UV-C radiation in a first mode when the doors are closed and the elevator cabin is unoccupied and cease emission of UV-C radiation in a second mode when the doors have opened. The sterilization devices 306 may remain in the second mode and may not resume emission of UV-C radiation until a predetermined time has elapsed since the opening and/or closing of the elevator doors and a person is not detected within the coverage zone. While in the second mode, the sterilization devices 306 may emit Far UV radiation as described. Furthermore, visible light assemblies of the sterilization devices 306 may be activated in the second mode and deactivated in the first mode.

In another example, the sterilization devices 306 may emit Far UV radiation in both the first mode and the second mode. The sterilization devices 306 may control the emission of UV-C radiation during only the first mode and/or control the activation of the visible light assemblies in only the second mode.

In some embodiments, each sterilization device 306 may operate independently. In some embodiments, a central processor of the elevator sterilization system 300 may control operation of the plurality of sterilization devices 306 by communicating with the control circuitry and/or processor thereof.

In embodiment utilizing a central processor, the elevator sterilization system 300 may regulate the sterilization devices to selectively emit radiation in specific areas within the elevator. For example, the central processor receives a signal that a new guest or person has entered the elevator (e.g., by a motion sensor of the sterilization devices 100 and/or a separate motion sensor of the system 300) and selectively activate specific sterilization devices 100 to cause radiation to be emitted in only the direction of the newly detected presence within the elevator. Furthermore, if the central processor receives a signal indicating a new presence within the elevator in addition to an existing presence (i.e., a first person has been in the elevator and a second person now enters the elevator), the central processor may selectively activate specific sterilization devices 100 to cause radiation to be emitted in only the direction of the newly sensed presence within the elevator.

In some embodiments, the system 300 further comprises a frame 308 for receiving and retaining the sterilization devices 302 in an array as shown in FIG. 3. However, the sterilization devices 100 may be affixed by any means as would be apparent to a person having an ordinary level of skill in the art. For example, the sterilization devices 100 may be affixed into existing cavities for downlights in an elevator cabin (e.g., by spring-loaded mounting clips 104 or other fixation means).

In some embodiments, the sterilization devices 306 further comprise a status-indicating device. In some embodiments, the status-indicating device may be one or more LEDs configured to emit light within the visible spectrum. In some embodiments, the status-indicating device may be operable to emit light in a plurality of visible wavelength ranges, each wavelength range corresponding to a status of the sterilization device 306. For example, the status-indicating device may indicate that irradiation is in progress by emitting light in a first visible wavelength range. In another example, the status-indicating device may indicate that irradiation is complete by emitting light in a second visible wavelength range. In another example, the status-indicating device may indicate an error or malfunction of the sterilization device 306 by emitting light in a third visible wavelength range. In some embodiments, the status of each sterilization device 306 as described may be individually indicated. In some embodiments, a plurality of sterilization device 306 or all sterilization devices 306 communicate with one or more shared status-indicating devices, e.g., a central status-indicating device of the elevator sterilization system 300.

In some embodiments, the elevator sterilization system 300 includes a power source in electrical communication with the control circuitry of each sterilization device 306 and/or each radiation assembly thereof and any additional electrical components, e.g., additional sensors, status-indicating devices, and the like. An electrical connection, e.g., a wired connection, may be used to connect the power source to the various components. In some embodiments, the power source may include a battery. In some embodiments, the power source may comprise a cable (not shown) configured to connect to a remote source of power via a plug or other connector at a remote end of the cable. In some embodiments, the power source is integrated with the sterilization device 306. In some embodiments, the sterilization device 306 is connected to a power source that services the elevator.

In some embodiments, the elevator sterilization system 300 further comprises an input device configured to control the one or more sterilization devices 306. The input device may perform any of the functions as described with respect to the elevator sterilization device 100. For example, the input device may be used by a user to set a sterilization mode and/or illumination mode of each sterilization device 306. In some embodiments, the modes of the sterilization devices 306 may be set uniformly, e.g., by communicating with the central processor. In some embodiments, the modes of the sterilization device 306 may be set individually, e.g., by communicating with the central processor and/or the control circuitry of each sterilization device.

In some embodiments, the sterilization devices 306 may be removable. For example, where a sterilization device 306 is not functioning properly or requires repair, the sterilization device 306 may be removed from the system 300 and/or replaced with another sterilization device 306.

In some embodiments, the system 300 may be fully or partially disposed within the elevator cabin 302, e.g., at the ceiling of the elevator cabin 302 as shown in FIG. 3. In some embodiments, the system 300 may be located adjacent the elevator cabin 302, e.g., directly above the elevator cabin 302 such that the coverage zones of the sterilization devices 306 extend into the elevator cabin 302.

In some embodiments, elevators may be constructed or manufactured with an integrated elevator sterilization system 300. For example, an elevator may be constructed with elevator sterilization devices 100 and/or an elevator sterilization system 300 as described integrated into the ceiling or another surface forming the elevator cabin 302.

The devices 100 and systems 300 described herein may be used in a variety of shared spaces, e.g., an entryway to an office, a public building, or any other building or structure utilizing an elevator. In some embodiments, devices 100 and systems 300 herein may be used in elevators in private buildings or structures, e.g., a home. In some embodiments, the devices 100 and systems 300 herein may be used in controlled spaces requiring a high degree of sanitization and/or sterile conditions. For example, the elevator sterilization devices and systems may be useful in hospitals, laboratories, testing facilities, care facilities, and the like. The devices 100 and systems 300 may also be useful in high traffic areas, e.g., public spaces or commercial spaces, and for large scale events, e.g., conferences, concerts, or other large gatherings in buildings or complexes. Non-limiting examples of public spaces where the devices 100 and systems 300 may be used include banks, hotels, airports, retail spaces, office spaces (e.g., rental or shared office spaces), residential complexes, shared conference or meeting spaces, event spaces, and the like.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A sterilization device for deactivating pathogens within an elevator cabin, the sterilization device comprising:
a housing defining an interior space and an aperture communicating with the interior space, and
one or more ultraviolet (UV) light sources arranged in the interior space and configured to emit UV light to a coverage zone within the elevator cabin via the aperture to deactivate pathogens therein;
one or more sensors configured to detect a living presence within the coverage zone;
a processor; and
a non-transitory, computer-readable medium storing instructions that, when executed, cause the processor to:
receive a first detection signal from the one or more sensors indicative of a living presence within the coverage zone,
operate the one or more UV light sources to emit UV light to the elevator cabin according to a first schedule in response to the one or more detection signals,
receive a second detection signal from the one or more sensors indicative of an absence of the living presence within the coverage zone,
after a first time delay from receiving the second detection signal, operate the one or more UV light sources to emit UV light to the elevator cabin according to a second schedule for a predetermined period of time, and
deactivate the one or more UV light sources after the predetermined period of time.

2. The sterilization device of claim 1, wherein the one or more UV light sources are configured to emit a sanitizing dose of UV light to the coverage zone within the predetermined period of time, wherein the sanitizing dose is configured to deactivate pathogens within the coverage zone.

3. The sterilization device of claim 1, wherein the one or more sensors comprise a passive infrared sensor configured to detect motion of the living presence.

4. The sterilization device of claim 1, wherein the housing further comprises one or more spring-loaded mounting clips configured to affix the housing within a light socket of an elevator cabin.

5. The sterilization device of claim 1, wherein the instructions that cause the processor to operate the one or more UV light sources according to a first schedule comprise instructions that, when executed, cause the processor to cycle the one or more UV light sources between a first period of about 70 seconds of emission and a second period of about 30 seconds of inactivity until the one or more light sources emit UV light for a total emission time.

6. The sterilization device of claim 5, wherein the total emission time is selected from the group consisting of about 12 minutes, about 18 minutes, about 24 minutes, and about 30 minutes.

7. The sterilization device of claim 5, further comprising an input device configured to receive input from a user related to the total emission time,
wherein the instructions, when executed, further cause the processor to:
receive the input from the user via the input device, and
set the total emission time based on the input.

8. The sterilization device of claim 1, wherein the instructions that cause the processor to operate the one or more UV light sources according to a second schedule comprise instructions that, when executed, cause the processor to cycle the one or more UV light sources between a first period of about 70 seconds of emission and a second period of about 30 seconds of inactivity until the one or more light sources emit UV light for a total of about 60 minutes during the predetermined time period.

9. The sterilization device of claim 8, wherein the predetermined period of time is about 86 minutes.

10. The sterilization device of claim 1, wherein the one or more UV light sources are selected from the group consisting of light-emitting diodes, mercury vapor discharge devices, laser diodes, pulsed xenon lasers, fiber lasers, and excimer lamps.

11. The sterilization device of claim 1, further comprising one or more visible light sources disposed on the housing and configured to emit visible light to the elevator cabin.

12. The sterilization device of claim 11, further comprising a translucent cap coupled to the housing over the one or more visible light sources,
wherein the visible light from the one or more visible light sources is configured to diffuse through the translucent cap and to the elevator cabin.

13. The sterilization device of claim 11, wherein the instructions, when executed, further cause the processor to:
activate the one or more visible light sources to emit the visible light in response to the one or more detection signals, and
after a second time delay from receiving the second detection signal, deactivate the one or more visible light sources.

14. The sterilization device of claim 13, wherein the first time delay is substantially equal to the second time delay.

15. A sterilization device for deactivating pathogens within an elevator cabin, the sterilization device comprising:
a housing defining an interior space and an aperture communicating with the interior space, and
one or more ultraviolet (UV) light sources arranged in the interior space and configured to emit UV light to a coverage zone within the elevator cabin via the aperture to deactivate pathogens therein;
one or more sensors configured to detect a living presence within the coverage zone;
a processor; and
a non-transitory, computer-readable medium storing instructions that, when executed, cause the processor to:
receive a first detection signal from the one or more sensors indicative of a living presence within the coverage zone,
maintain the one or more UV light sources in a deactivated state in response to the one or more detection signals,
receive a second detection signal from the one or more sensors indicative of an absence of the living presence within the coverage zone,
after a first time delay from receiving the second detection signal, activate the one or more UV light sources to emit UV light to the elevator cabin according to a schedule for a predetermined period of time, and
deactivate the one or more UV light sources after the predetermined period of time.

16. The sterilization device of claim 15, further comprising:
one or more visible light sources disposed on the housing and configured to emit visible light to the elevator cabin; and
a translucent cap coupled to the housing over the one or more visible light sources,
wherein the visible light from the one or more visible light sources is configured to diffuse through the translucent cap and to the elevator cabin.

17. The sterilization device of claim 16, wherein the instructions, when executed, further cause the processor to:
activate the one or more visible light sources to emit the visible light in response to the one or more detection signals, and
after a second time delay from receiving the second detection signal, deactivate the one or more visible light sources.

18. The sterilization device of claim 15, wherein the first time delay is substantially equal to the second time delay.

19. The sterilization device of claim 15, wherein the instructions that cause the processor to operate the one or more UV light sources according to a schedule comprise instructions that, when executed, cause the processor to cycle the one or more UV light sources between a first period of about 70 seconds of emission and a second period of about 30 seconds of inactivity until the one or more light sources emit UV light for a total of about 60 minutes during the predetermined time period.

20. The sterilization device of claim 19, wherein the predetermined period of time is about 86 minutes.

21. An elevator sterilization system for deactivating pathogens, the elevator sterilization system comprising:
an elevator cabin including one or more doors;
one or more sterilization devices, each sterilization device comprising:
a housing defining an interior space and an aperture communicating with the interior space, and
one or more ultraviolet (UV) light sources arranged in the interior space and configured to emit UV light to a coverage zone within the elevator cabin via the aperture to deactivate pathogens therein;
one or more sensors configured to detect a living presence proximate the elevator cabin;
a processor; and
a non-transitory, computer-readable medium storing instructions that, when executed, cause the processor to:
receive one or more first detection signals from the one or more sensors indicative of a living presence within the coverage zone of a first set of the one or more sterilization devices,
operate the one or more UV light sources of the first set to emit UV light to the elevator cabin according to a first schedule in response to the one or more first detection signals,
receive one or more second detection signals from the one or more sensors indicative of an absence of the living presence within the coverage zone of the first set,
after a first time delay from receiving the second detection signal, operate the one or more UV light sources of the first set to emit UV light to the elevator cabin according to a second schedule for a predetermined period of time, and
deactivate the one or more UV light sources after the predetermined period of time.

22. The system of claim 21, wherein each sensor is associated with a sterilization device of the one or more sterilization devices, wherein the sensor is configured to detect the living presence within the coverage zone.

23. The system of claim 21, wherein the instructions that cause the processor to operate the one or more UV light sources of the first set according to a first schedule comprise instructions that, when executed, cause the processor to cycle the one or more UV light sources of the first set between a first period of about 70 seconds of emission and a second period of about 30 seconds of inactivity until the one or more light sources emit UV light for a total emission time,
wherein the total emission time is selected from the group consisting of about 12 minutes, about 18 minutes, about 24 minutes, and about 30 minutes.

24. The system of claim 21, further comprising a frame comprising a plurality of recesses, each recess configured to receive a sterilization device of the one or more sterilization devices to affix the sterilization device to the frame.

25. The system of claim 21, wherein at least one of the one or more sensors comprise one or more light sensors configured to detect one or more changes in ambient light in the elevator cabin associated with opening of the one or more doors,
wherein the one or more first detection signals and the one or more second detection signals are associated with the one or more changes in ambient light.

26. The system of claim 21, wherein each sterilization device further comprises:
one or more visible light sources disposed on the housing and configured to emit visible light to the elevator cabin; and
a translucent cap coupled to the housing over the one or more visible light sources,
wherein the visible light from the one or more visible light sources is configured to diffuse through the translucent cap and to the elevator cabin.

27. The system of claim 26, wherein the elevator cabin further comprises a titanium oxide coating on one or more surfaces of the elevator cabin.

28. The system of claim 27, wherein the one or more visible light sources of each sterilization device comprise a blue light pump having a peak wavelength of about 435 nm,
wherein the titanium oxide coating reacts with the visible light from the one or more visible light sources to induce photocatalytic effects on the one or more surfaces.
